(19) United States
(12) Patent Application Publication
(10) Pub. No.: US 2010/0010365 A1
(43) Pub. Date: Jan. 14, 2010

(54) Apparatus for analyzing brain wave

(75) Inventors: Motoyasu Terao, Hinode (JP); Shigeru Oho, Tokyo (JP); Yoshitaka Sasago, Tachikawa (JP)

Correspondence Address: Juan Carlos A. Marquez;c/o Stites & Harbison PLLC, 1199 North Fairfax Street, Suite 900, Alexandria, VA, 22314-1437, US

(73) Assignee: Hitachi, Ltd.

(21) Appl. No.: 12/453,267

(22) Filed: May 5, 2009

(30) Foreign Application Priority Data
Jul. 11, 2008 (JP) 2008-180881

(51) Int. Cl. A61B 5/0476

(52) U.S. Cl. 600/544

(57) ABSTRACT

To increase intensity of the brain wave signal for detection. Provided is an apparatus for analyzing a brain wave which is installed on a vehicle comprising: a detection unit for detecting the brain wave signal, and separating and analyzing the detected brain wave signal; a discrimination unit for generating a control signal according to an intensity of the brain wave signal analyzed by the detection unit; a processing control unit for controlling subsequent processing according to a type of each of the plurality of control signals produced by the discrimination unit; at least one brain wave signal induction unit for generating a graphic inducing a predetermined type of the brain wave; and a display for displaying the generated graphic according to the received signal from the brain wave signal induction unit.

### CLAIM OF PRIORITY

The present application claims priority from Japanese patent application 2008-180881 filed on Jul. 11, 2008, the content of which is hereby incorporated by reference into this application.

### BACKGROUND OF THE INVENTION

This invention relates to apparatus for analyzing a brain wave which is installed on a vehicle, and more particularly, to a storage and control device using a brain wave.

Among onboard devices for motor vehicles, a conventional car navigation device, for example, includes a global positioning system (GPS) sensor for detecting the location of the vehicle, a velocity sensor, a data storage unit for storing map data, a key input unit including various keys for inputting control signals according to operations, and a display unit for displaying a map. A user can operate keys on the key input unit to set a destination, and to check a route and a vehicle location during travel.

In place of the key operations on the input unit, JP 2003-248541 A discloses control devices using a brain wave. According to a description thereof, on a conventional navigation device, in order to operate keys on the navigation device during driving, it is necessary to move the line of sight from a road to an input unit of the navigation unit, which may lead to an accident. Therefore, according to JP 2003-248541 A, in place of the key operation, though an operation based on voice recognition has been proposed, due to the accuracy of the voice recognition and the like, eventually the key operation becomes necessary. Moreover, as JP 2003-248541 A describes, there is a description that a car navigation system is operated and a part of driving is carried out using a brain wave. Moreover, U.S. Pat. No. 7,127,283 B2 and US 2004/0098193 A1 similarly disclose control of vehicle onboard devices using a brain wave signal.

### SUMMARY OF THE INVENTION

However, according to above-mentioned JP 2003-248541 A, U.S. Pat. No. 7,127,283 B2, and US 2004/0098193 A1, though the navigation device is operated by comparing a brain wave pattern registered in advance and a detected brain wave signal with each other, those documents do not describe how the intensity of the brain wave signal is increased for detection, and how the determination can be surely made.

This invention has been made in view of the above-mentioned problems, and an object of this invention becomes apparent later.

A representative aspect of this invention is as follows. That is, there is provided an apparatus for analyzing a brain wave which is installed on a vehicle comprising: a detection unit for detecting the brain wave signal, and separating and analyzing the detected brain wave signal; a discrimination unit for generating a control signal according to an intensity of the brain wave signal analyzed by the detection unit; a processing control unit for controlling subsequent processing according to a type of each of the plurality of control signals produced by the discrimination unit; at least one brain wave signal induction unit for generating a graphic inducing a predetermined type of the brain wave; and a display for displaying the generated graphic according to the received signal from the brain wave signal induction unit.

A description is now given of effects of this invention. For example, according to this invention, an operation can be carried out without affecting other operations such as driving a motor vehicle which need to be carried out almost continuously. An operation can be carried out without a delay. An operation can surly be carried out. Training is possible for an operation. Depending on proficiency achieved by the training, an optimal operation is selected. A strong light source or the like is not present, and, thus, a safe operation is provided. An operation may be conducted in a non-contact manner. Influence of hairs and, depending on installed locations of detectors, the cranial bone can be avoided.

### BRIEF DESCRIPTION OF THE DRAWINGS

The present invention can be appreciated by the description which follows in conjunction with the following figures, wherein:

FIG. 1 is a block diagram illustrating a configuration of a storage and control device according to a first embodiment of this invention;

FIG. 2 is a block diagram illustrating an internal configuration of a storage unit of FIG. 1;

FIG. 3 is a block diagram especially illustrating a configuration of a display unit according to the first embodiment of this invention;

FIG. 4 is a flowchart illustrating a control method using a brain wave according to the first embodiment of this invention;

FIG. 5 is an explanatory diagram illustrating a hierarchical structure of options according to the first embodiment of this invention;

FIG. 6 is an explanatory diagram illustrating how to generate a control signal using parts of a displayed map of a car navigation system according to the first embodiment of this invention;

FIG. 7 is an explanatory diagram illustrating how to generate a control signal using parts of a displayed map of the car navigation system according to the first embodiment of this invention;

FIG. 8 is an explanatory diagram illustrating how to generate a control signal using parts of a displayed map of the car navigation system according to the first embodiment of this invention;

FIG. 9 is an explanatory diagram illustrating how to generate a control signal using parts of a displayed map of the car navigation system according to the first embodiment of this invention;

FIG. 10 is an explanatory diagram illustrating how to generate a control signal using parts of a displayed map of the car navigation system according to the first embodiment of this invention;

FIG. 11 is an explanatory diagram illustrating how to generate a control signal using parts of a displayed map of the car navigation system according to the first embodiment of this invention;

FIG. 12 is an explanatory diagram illustrating how to generate a control signal using parts of a displayed map of the car navigation system according to the first embodiment of this invention; and

FIG. 13 is an explanatory diagram illustrating an arrangement of devices according to the first embodiment of this invention.

### DETAILED DESCRIPTION OF THE PREFERRED EMBODIMENT

A description is now given of an embodiment of this invention. A brain wave of the human is formed of frequency bands ranging from 0 Hz to several hundreds Hz. The embodiment to be described is an example in which vehicle onboard devices such as a car navigation device, a car audio device, and a car video device are controlled by the brain wave. The control for those devices often includes both control by switches concentrated on a steering wheel for driving and control through a touch panel conventionally.

On the vehicle onboard device according to this invention, options for the control are shown on a display as graphics inducing a brain wave, and selection is carried out by viewing them, imagining a motion of a hand or a foot, or actually moving it. By showing the graphics inducing the brain wave on the display, the intensity of the brain wave can be increased. Moreover, by showing as a bar chart predetermined components of a brain wave induced by seeing the option on the display, a driver can check the own brain wave, thereby intentionally control consciousness to increase the intensity of the brain wave.

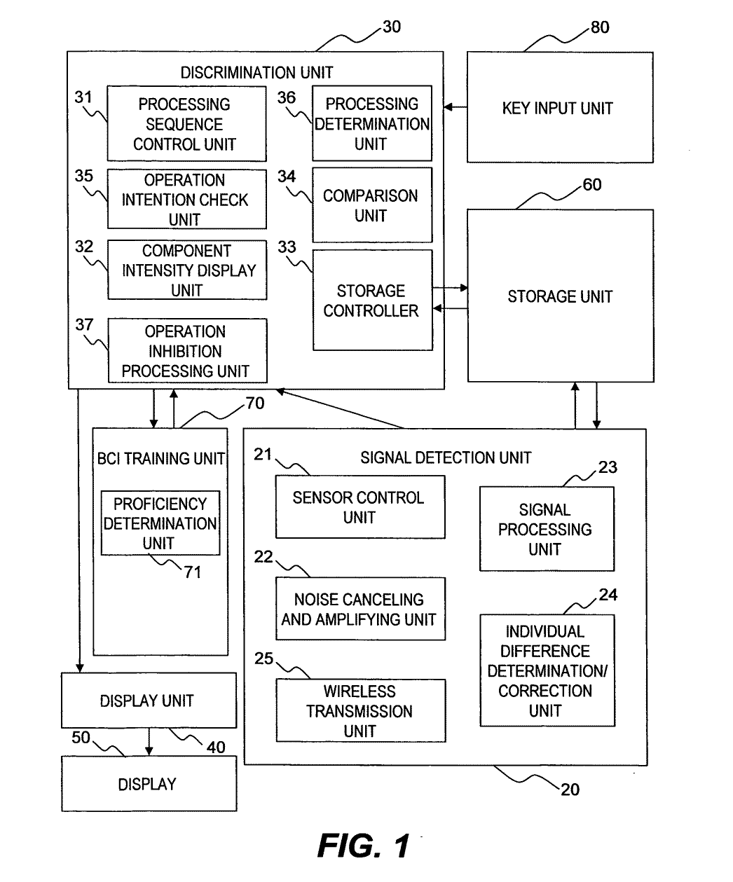

FIG. 1 is a block diagram of a vehicle onboard device according to this invention. The vehicle onboard device includes a signal detection unit 20, a discrimination unit 30, a display unit 40, display 50, and a storage unit 60, and optionally includes a BCI training unit 70 and a key input unit 80 according to necessity. As configurations of the respective units, the discrimination unit 30 includes a processing sequence control unit 31, a component intensity display unit 32, a storage controller 33, a comparison unit 34, an operation intention check unit 35, and a processing determination unit 36. The processing sequence control unit 31 controls a flow for the entire vehicle onboard device such as a processing sequence for specifically carrying out an instruction by the driver. The component intensity display unit 32 generate data for displaying component intensities of the brain wave detected by the signal detection unit 20 on the display, and enables the operator to confirm that the intensities of components are increased as the operator intends. Though an intensity display of one component of a brain wave is known, it is preferable to decompose a brain wave into components, and then to simultaneously or sequentially display intensities of the respective components as a bar chart for comparison. In addition to the length of bars of the bar chart, the intensities are preferably represented by the size or area of graphics. The storage controller 33 controls read and write of waveforms from and to the storage unit 60. The discrimination unit 30 compares a waveform read from the storage unit 60 and a waveform processed by the signal detection unit 20 with each other, selects one of multiple options according to a result of the comparison, and notifies the processing sequence control unit 31 of the selected option.

The signal detection unit 20 includes a sensor control unit 21 for controlling an angle and a position of sensors when a relationship in position between the sensors and the head can be changed, a noise canceling and amplifying unit 22 for canceling noises of and amplifying a received signal, a signal processing unit 23 for extracting signals significant for the control, and correcting distortions, and an individual difference determination/correction unit 24 for determining and correcting differences of the obtained signal among individuals, thereby producing a signal used for the control.

The discrimination unit 30 includes an operation inhibition processing unit 37 according to necessity. The operation inhibition processing unit 37 according to this embodiment corresponds to a component which inhibits operations on a conventional car navigation device while a vehicle is traveling. However, a brain wave is used, danger is hardly generated even during traveling, and the operation inhibition processing unit 37 is different from the conventional operation inhibition processing unit 37 in that inhibited processing is limited only to processing requiring a plurality of predetermined steps, for example, the number of steps equal to or larger than five. This threshold step number used by the inhibited processing is preferably determined according to the proficiency determined by the BCI training unit 70. Moreover, according to an individual difference in the detected brain wave waveform, an identification (personal authentication) is carried out, and in a case of which the identification is not certified, inhibition processing for preventing driving by an unauthorized person or theft is carried out. The identification according to the detection of an individual difference in the brain wave is effective as at least one of means for personal identification in operation of the automatic telling machine in addition to driving a motor vehicle.

The BCI training unit 70 includes a proficiency determination unit 71 for determining proficiency in operation using a brain wave, for example, according to a period of time from the start of operation processing until operating brain wave reaches a predetermined intensity or a frequency of operation error.

The vehicle onboard device according to this invention additionally includes a display unit 40, a storage unit 60, and a key input unit 80. Thought the key input unit 80 preferably includes a push button or a group of push buttons, the key input unit 80 may be a device such as a keyboard or a touch panel that the operator can use to input intention by means of something other than the brain wave.

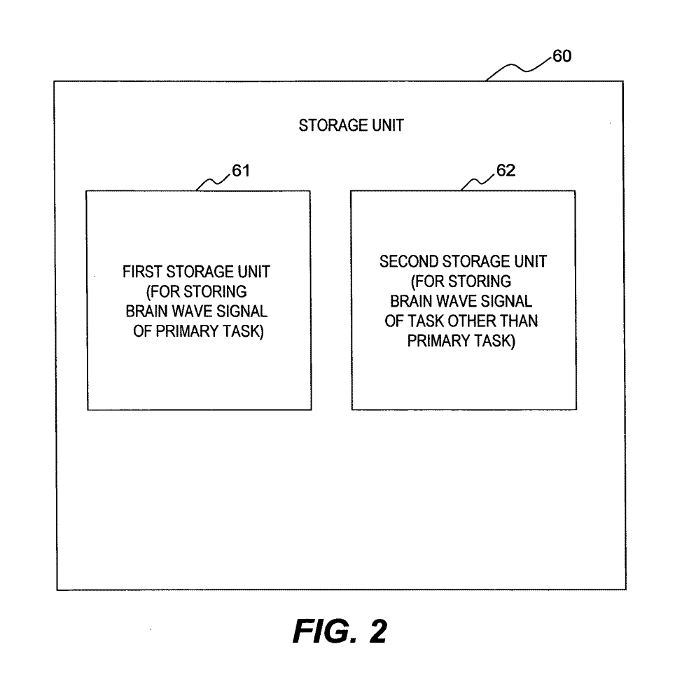

The storage unit 60 is more preferably divided into a first storage unit 61 for storing a brain wave signal of primary task, and an second storage unit 62 for storing a brain wave signal of task other than the primary task, as illustrated in FIG. 2. For example, in a case of a motor vehicle, brain waves relating to the driving are stored in the first storage unit 61, and brain waves relating to operations such as operations on the car navigation device, and operations on the audio and video devices are stored in the second storage unit 62. In this way, by storing waveforms of brain waves relating to the primary task, in a case of which the vehicle onboard device is operated during driving, and one of options is selected by the comparison unit 34, it is possible to distinguish a brain wave relating to the operation of the vehicle onboard device from brain waves relating to the primary task, and thus to surely operate the vehicle onboard device.

Moreover, by providing the storage unit 60 with a sufficient capacity, at least a part of a brain wave which is detected by the signal detection unit 20, and is processed by the signal processing is preferably not deleted and is accumulated in the storage unit 60 after the brain wave is used for the control. Consequently, the accumulated brain waves can be used for obtaining representative waveforms such as averaging and feature extraction, thereby increasing reliability in the detection and reliability in the extraction of individual differences. This part is preferably provided as an independent area as a waveform accumulation unit.

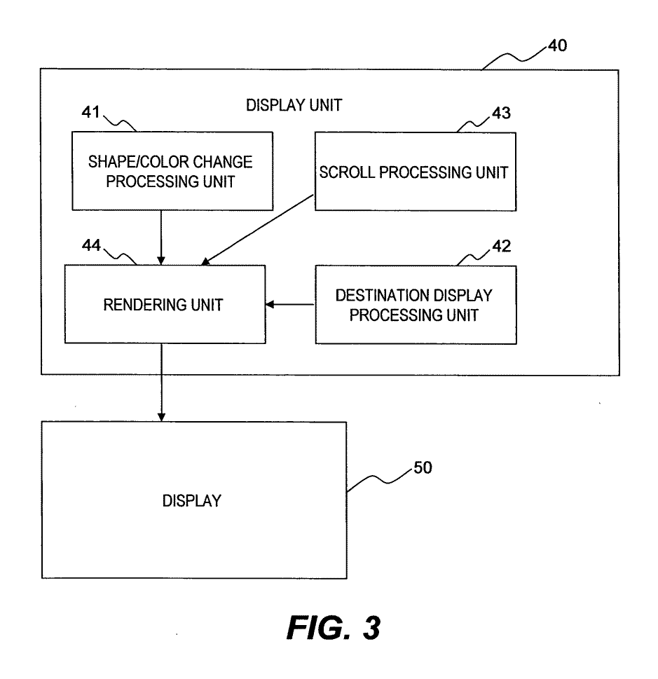

FIG. 3 shows detail of the display unit 40 of the vehicle onboard device of FIG. 1. The display unit 40 includes a shape/color change processing unit 41, a destination display processing unit 42, a scroll processing unit 43, and a rendering unit 44. The shape/color change processing unit 41 changes shapes and colors of a screen on the display for promoting induction of a brain wave for driving a screen. A screen display for promoting the induction of the screen driving brain wave is a screen display which is different from a normal screen display, and is partially changed in shape and color. For example, when a part close to an edge of the display area, such as a map or characters displayed on a lower right portion, is expanded only in the vertical direction, or in the horizontal direction, or is deformed in a manner other than zooming in/out, viewing this part will produce a sense of abnormality, and, thus, a brain wave able to scroll the screen up/down and leftward/rightward or able to switch the screen tends to be induced. The color change includes a change in background color, a change in color of characters, negative/positive inversion, a display in a complementary color, a display used when headlights are on while the headlights are actually off, and a display used when the headlights are off while the headlights are actually on. Even when the change in shape is zooming in/out, though the reliability of the operation decreases, the operation may be possible. Though a boundary between an area in which a display is deformed and an area in which a display is normal preferably transitions continuously, a change as a step function may be possible. Preferably, a plurality of areas in which a display is deformed exist, and positions and manners of the change in shape/color represent options for the control. The destination display processing unit 42 carries out a display for setting destination in a case of which a destination is set. The scroll processing unit controls scroll of the display screen. Signals output from those plurality of processing units 41, 42, 43 are finally processed for display by the rendering unit 44, and is supplied to the display.

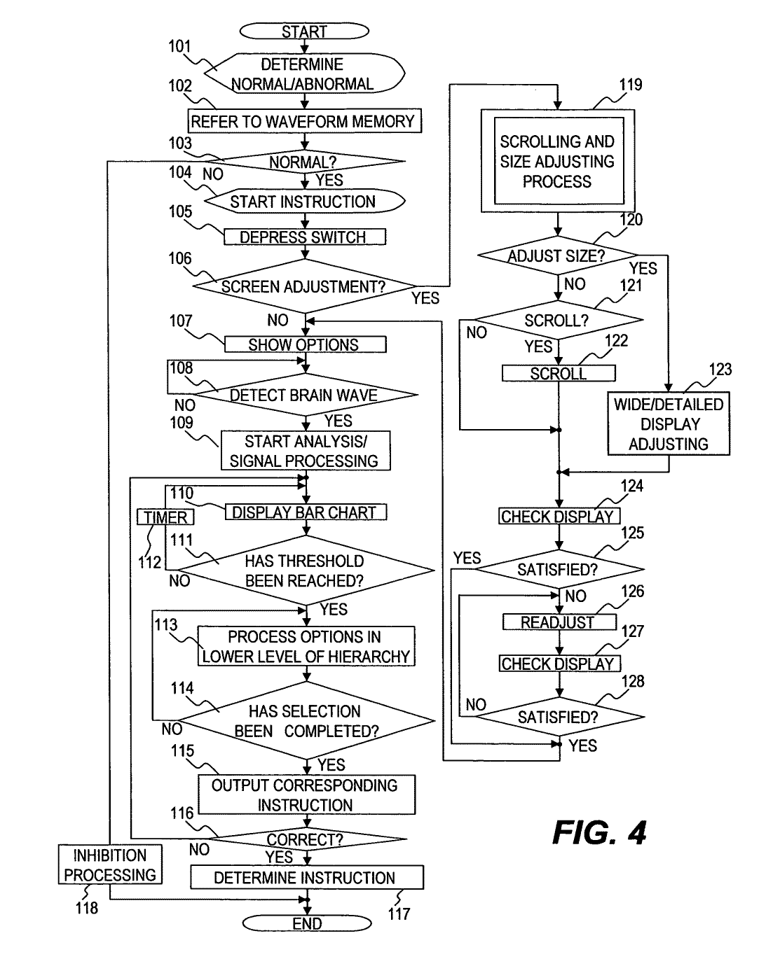

FIG. 4 illustrates an example of a flowchart for control. It should be noted that an overall flow is controlled by the processing sequence control unit 31. First, when the operator turns on the key or the like to turn on the vehicle onboard device, or gives an instruction to start operation of the vehicle onboard device by key input, the operation inhibition processing unit 37 determines whether the state of the brain wave is normal or abnormal (100). The operation inhibition processing unit 37 receives the brain wave signal from the signal detection unit 20, and causes the comparison unit 34 to compare the brain wave signal with brain waves held in the storage unit 60 (102), thereby detecting whether the state of the brain wave is abnormal (103). In a case of which the operator is drunken, or is likely to fall asleep, the comparison unit 34 detects abnormality such as frequency components different from those in the normal state in the brain wave and a slow response to a change in the display screen. Though the description is given of the case in which abnormality is detected according to abnormal waveforms stored in the storage unit 60, abnormality may be determined by a logic circuit. In a case of which abnormality is detected, the operation inhibition processing unit 37 proceeds to drive inhibition processing such as safe stop of traveling or stop of an engine (inhibiting operation) (118). In a case of which the state of the brain wave is normal, the operator can operate the motor vehicle or the vehicle onboard device such as the car navigation device (104). On this occasion, when the operator operates the car navigation device, the operator shows an intention of starting instruction by means of a switch in a vehicle cabin, such as an input on a key installed on a steering wheel (depressing the switch, for example) (105). As a result, the processing sequence control unit 31 transmits an instruction to the display unit 40, and options for the operations are shown in the display (106, 107). When the operator sees a display corresponding to an option which the operator wants to select, thinks about the option to select, or imagines a behavior corresponding to this option, a predetermined brain wave appears, and, thus, the processing sequence control unit 31 waits for detection of the brain wave (108). The brain wave appears independently of the operation of the navigation device. Thus, as in this invention, by carrying out the key input to show whether or not to control the navigation device by means of the brain wave, it is possible to prevent the navigation device from malfunctioning when the operator does not intend to operate the navigation device. The signal detection unit 20, when the brain wave is detected, starts, in the signal detection unit 20 illustrated in the block diagram, analysis such as noise canceling, amplification, signal processing, and correction, and notifies the discrimination unit 30 of a result of the analysis (109). When the signal detection unit 20 detects the brain wave, the component intensity display unit 32 of the discrimination unit 30 instructs the display unit 40 to show decomposed brain wave components as a bar chart (110-112). As a result, the operator can intentionally intensify a subject component.

Then, the comparison unit 34 searches for, by comparing the detected brain wave signal with waveforms read from the storage unit 60, a corresponding option. On this occasion, it is preferable to bypass waveforms caused by the primary task, which is not illustrated in the drawings. When a corresponding waveform is found, an instruction (such as a control signal or a command) is output from the comparison unit 34 to the processing sequence control unit 31 (115). On this occasion, the processing sequence control unit 31 shows contents of the instruction before determining the instruction, and, when the shown contents are wrong, repeats the processing from the display of the bar chart (116, 117). For example, when the determined instruction is an instruction for adjusting the screen (106), and a map is shown on the screen, the processing sequence control unit 31 enters a process for scrolling and adjusting the size (wide/detailed map display). In the flowchart for this process, though a flow from the detection of the brain wave to the determination of the instruction is not explicitly illustrated, and is represented as “ADJUST SIZE?” and “SCROLL?”, it is preferable to employ the brain wave detection for determination of those instructions as well (119-128).

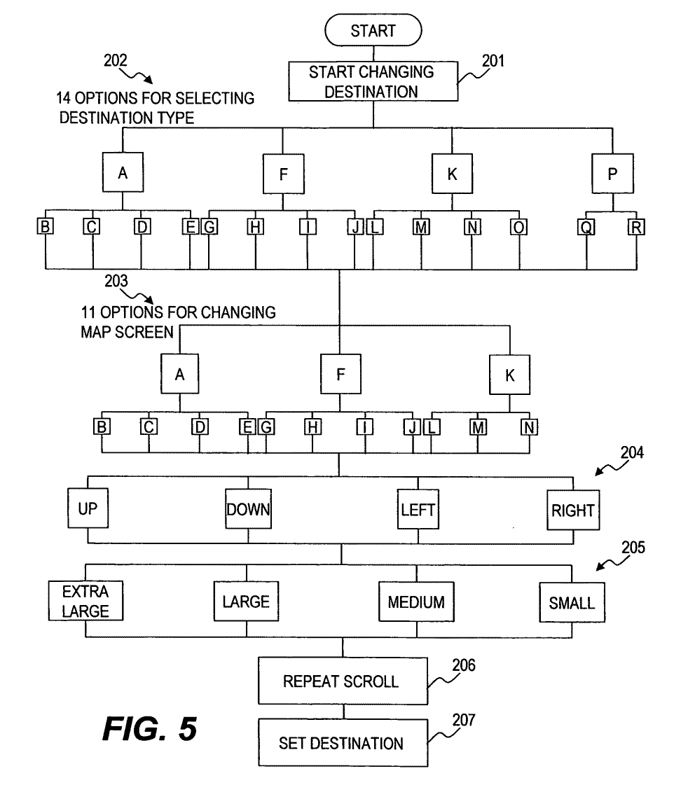

FIG. 5 illustrates a hierarchical structure of the portion for processing the options in the flowchart in FIG. 4. As described above, when the operator depresses the instruction start switch to start selecting an operation, the options are shown (201), and, for more reliable selection by means of the brain wave, the operation is preferably selected not from a large number of options at a time, but from at most six options, from four options in FIG. 5 (202). In the example illustrated in FIG. 5, there are fourteen types of destination including B to R, and hence, the destinations are arranged into two levels or more of hierarchy as illustrated in FIG. 5, and are selected sequentially. A group at the second level of hierarchy is arranged, for example, in the alphabetical ascending order of the initials of destination type or in order from the north, and do not entirely correspond to conventional options such as names of prefectures (203). A next selection of up, down, left, or right is selection for a scroll direction (204). A next selection is a distance of the scroll (205). Those selections are repeated according to necessity (206), and when the center of the map reaches the destination, the operator selects to set the destination (207). In this way, by arranging the options into a hierarchical structure, it is possible to restrict types of brain wave to be detected/discriminated at a time, resulting in more reliable discrimination.

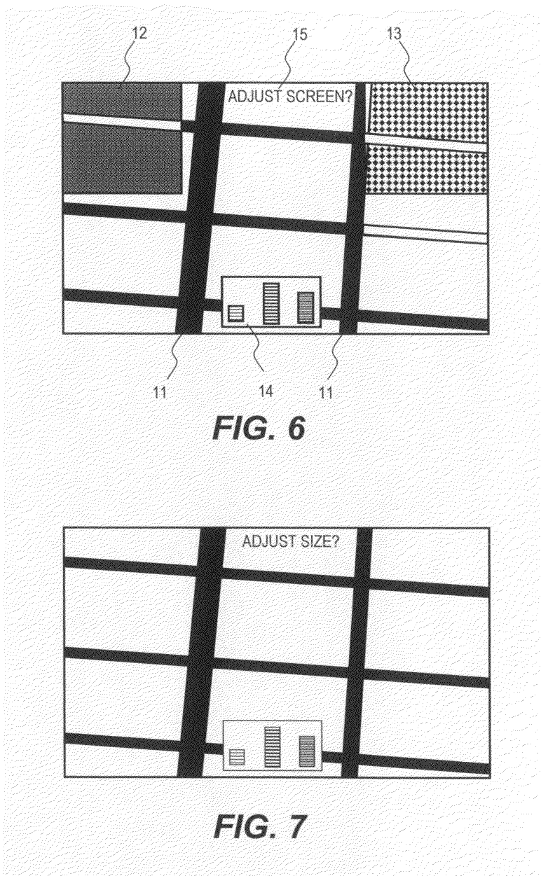

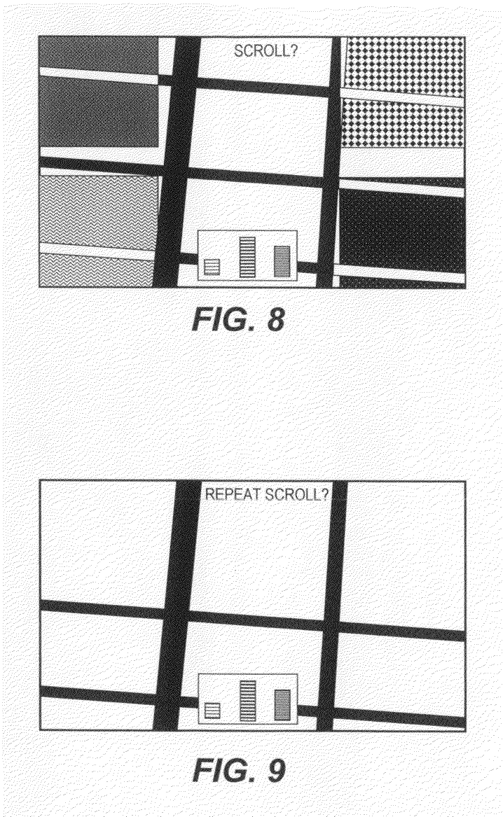

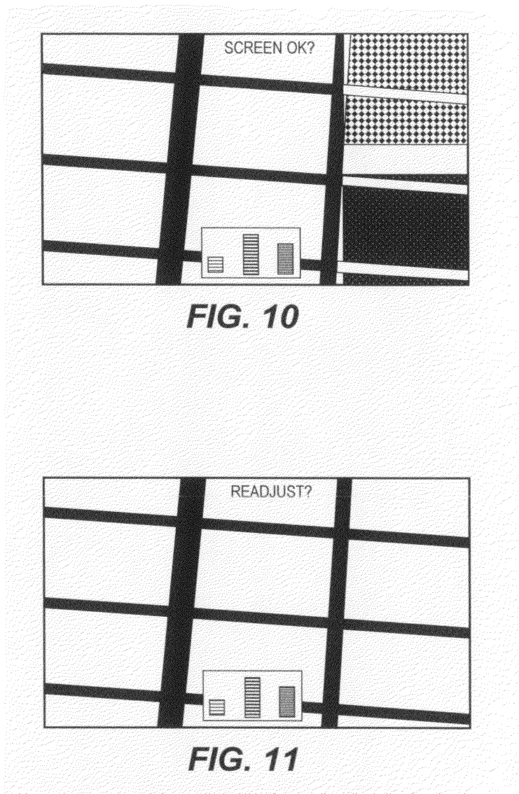

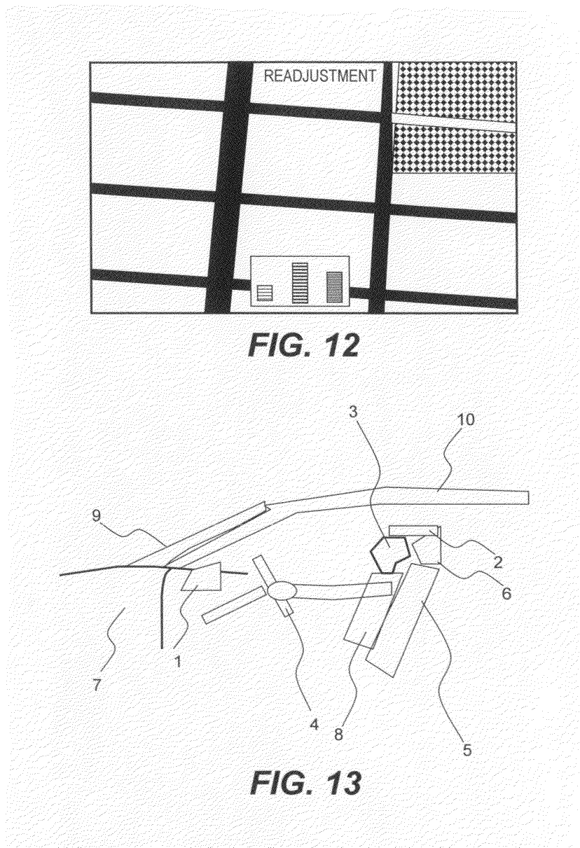

A description is now given of the switching between the “wide display” and the “detailed display” of the map of the car navigation system, and when the operator wants the switching, and, as in the example illustrated as the flowchart of FIG. 4, depresses the push button switch installed on the steering wheel for starting the brain wave sensing, the process proceeds to a step for determining whether or not the screen operation is necessary. As illustrated in FIG. 6, parts representing options which requires or do not require the screen operation are displayed on a map display screen. In the example illustrated in FIG. 6, the parts which is changed or inverted in color by the shape/color change processing unit 41 are shown at an upper left location 12 and an upper right location 13. On the parts in which the color for display is changed or inverted, characters “YES” and “NO” may be shown, but the operator may have leaned in advance correspondence between “YES” and “NO” and positions such as left and right or colors of the display patterns. In FIG. 6, parts appearing as a grid are roads 11, and buildings and the like other than the roads are omitted. The operator, by viewing and selecting any one of them, branches to any one of flows corresponding to “YES” and “NO”. In a step of the branched destination, the operator may similarly look at and select an option on the screen, but changing the way of selection tends to increase the possibility of successful selection. For example, in place of the selection by viewing, the selection is carried out by imagining a motion of a hand or a foot, or actually moving it. FIG. 7, FIG. 9 and FIG. 11 are screens corresponding thereto. On a motor vehicle equipped with an automatic transmission, a driver usually uses the right foot for operating the accelerator or the brake, and may use the left foot for applying a foot parking brake, but does not use the left foot while driving or operating the car navigation system. Thus, the operator preferably imagines a motion of the left foot, or even actually moves the left foot. On a motor vehicle in which the brake and accelerator are operated by the left foot, it is preferable to imagine a motion of the right foot. Further, in a next step, the way of selection may be switched back to the display on the screen. In the example in the drawings, parts representing options are shown at four corners. By showing an option at the center on the left end, right end, top end, or bottom end, options may be shown at five or more locations. When the operator looks at the display part at one of the four corners for more than 0.5 seconds, a bar chart 14 indicating the intensity of a brain wave corresponding to the option is shown on a part of the screen. When a bar does not exceed a threshold, by viewing the part again, or imagining the part, it is possible to increase the intensity.

Then, for example, in order to select an audio section of the car navigation system, when the operator depresses the push button installed on the steering wheel for starting the brain wave sensing, a plurality of graphics representing options for selecting a type of operation by means of the brain wave are shown. The graphics include characters specifically indicating the options. When the operator looks at a graphic indicating an audio operation for more than 0.5 seconds, a bar chart indicating the intensity of a brain wave corresponding to the option is shown on a part of the screen. When a bar does not exceed a threshold, by viewing the graphic again, or imagining the graphic, it is possible to increase the intensity. When the graphic representing “audio operation” is selected, then, graphics representing options for selecting “radio”, “television”, “HDD”, or “CD” are shown. In response to the display, as described above, “HDD” is selected. “CD” may be selected. By repeating this operation, it is possible to listen to desired music. When options and the chart showing the brain wave intensity are shown not on the display of the car navigation system, but in a neighborhood of a display showing the vehicle speed, the operation is possible by slightly moving the eye sight from the front in the traveling direction, resulting in a further increase in safety. Graphics representing the options may be four to six graphics including a photograph or illustration of a face of an idol or a TV comedian, a cat and a centipede, a graphic of a smile in a circle and an equation, and combinations thereof. On a part of those graphics, characters such as “WIDE” and “DETAILED” indicating the options are written. By alternately combining the selection method by means of imagining a movement of a hand or foot, for example, with the graphics, more reliable selection can be carried out. As described above, regardless of whether the operation is intended for the audio device or the map, it is preferable, considering the characteristic of the selection using the brain wave, that the options are more finely divided and arranged into a hierarchical structure than that in a case of a touch panel, and the selection is repeated for groups including at most six, preferably four, more preferably two options.

A relationship between visual recognition of graphics and the brain wave has not been sufficiently clarified, and thus, as the clarification progresses in the future, it is expected that graphics applicable to quick and reliable selection are identified, and present graphics are preferably replaced by the identified graphics.

As a selection other than the selection by means of graphics, for example, a change in the brain wave caused by imagining a motion of the hand or foot, actually moving the eyeball, or moving fingers may be used. Which finger is moved may be identified in the future based on a change in the brain wave caused by the motion of the moved finger, which is presently impossible, and a selection may be made from as many as ten options.

The control and storage devices of this invention excluding sensors and the key input unit 80, but including a wireless transmission unit 25 and a power supply unit according to necessity, are most preferably implemented on one semiconductor chip, and the devices excluding the power supply unit are second most preferably implemented on one chip. Third most preferably, the devices are implemented on a single circuit board.

FIG. 13 illustrates an arrangement of a brain wave sensor when the control device according to this invention is installed on a motor vehicle. Reference numeral 1 denotes a display, and the configuration illustrated in FIG. 1 is integrated into the display. Reference numeral 2 denotes the brain wave sensor. Reference numeral 3 denotes a head of a driver (operator); and 4, a steering wheel on which the key input unit 80 is installed. Reference numeral 5 denotes a seat back; and 6, a headrest. Reference numeral 7 denotes a front portion of a vehicle body; and 8, torso of the driver. Reference numeral 9 denotes a front windshield; and 10, a ceiling. According to this embodiment, the brain wave sensor 2 is installed on the headrest 6. The brain wave sensor 2 may be formed into a helmet, may then be worn on the head 3, and electrodes may be attached to the scalp. Alternatively, the brain wave sensor 2 may be formed into a head phone, and the electrodes may be pressed against the scalp, or the brain wave sensor 2 may be hung from the ceiling 10 of the vehicle body. The brain wave sensor 2 preferably carries out the detection in a non-contact manner. In this case, it is preferable to provide position control so that a relative position between the sensor and the head is maintained.

Though, for detecting the brain wave, several sensor electrodes are generally attached to the scalp, there has been a report that an electrocardiogram acquired by detecting a similar electric signal appearing on the human skin can be realized in a non-contact manner. It is considered that the brain wave can be detected in a non-contact manner by removing noise components, processing signals, and amplifying the signals. The non-contact detection is carried out using at least two non-contact electrodes, an amplifier having a high input impedance, a notched filter, and the like. While the electrodes are provided on a chair (such as a seat back and a seat pan) for the electrocardiogram, the electrodes are preferably provided on a headrest (pillow) or a component hung from the ceiling. As another method, according to research conducted by the same laboratory, intrabody communication is possible, and thus, it is theoretically possible to transmit a signal from a part to which a brain wave sensor of a headphone type is attached through the human body by means of the intrabody communication, and to detect the brain wave at a chair portion.

A configuration in which the brain wave signal is detected on the scalp or in a neighborhood of the brain cortex, and is amplified and processed by a microcomputer in a neighborhood thereof, and is wirelessly transmitted is free from trouble of wiring, and is thus preferable.

On at least one control screen, contents of control may be sequentially displayed, or simultaneously displayed side by side, and an operator may show an intention of whether to approve or decline visually recognized control contents by means of thoughts (thoughts are defined to include images of moving the hand or foot according to this invention), and control may be carried out according to resulting detected brain wave.

A part of the display screen may serve as a screen corresponding to at least one of zooming in, zooming out, screen scrolling, and destination setting, and, by tracking the part by the eyes, at least one of corresponding operations may be realized according to necessity.

When the detection of the brain wave is uncertain due to strong external noise or the like, detection of a blood volume on the surface of brain cortex by irradiating light/detecting reflected light, which is referred to as NIRS or optical topography, or voice recognition, which is reported to be in use for control of vehicle onboard devices, may be employed at the same time, and when the same determinations are made by both of them, a control operation may be determined.

In the embodiment, this invention is described while the navigation device is exemplified as a subject of the control provided by the control device employing the brain wave, but this invention may be applied to a control device controlling any control subject in addition to the navigation device. In a vehicle cabin, in addition to the navigation device, an air conditioner and an audio device are installed. Therefore, those devices may be controlled by means of the brain wave. Types of control on the air conditioner and the audio device include temperature adjustment for the air conditioner, and tuning to a broadcast channel, selection of a song on a CD or the like to be played, and volume adjustment for the audio device.

The process for determining whether a driver is sleeping or drunken may be independent of the device control process, and more precise determination is provided by also employing other detected data such as the heart rate.

Moreover, in place of a keyboard or a mouse, the computer can be controlled by the brain wave. A patient can use a communication device installed bedside a pillow to transmit requests of the patient to a remote person.

Further, an electronic device to be controlled may be combined with a mechanical device, and such combinations include a robot arm, a robot foot, an entire robot providing various functions, and an electronic wheel chair.

The vehicle onboard device controlled by the brain wave signal has been described based on the embodiment, and the brain wave is mainly produced as a result of a large number of overlapping electronic potentials generated on the cell body and axon of neurons as a result of activities of the neurons in the brain, and is influenced, in the case of detection on the scalp, by non-isotropy of the dielectric constant of the cranial bone. The activities of the neurons are not completely independent of each other, and are often synchronized. As a result, the activities may be detected as a relatively large signal. The activities vary according to the location on the brain, and hence a large number of sensors installed on a helmet increase the degree of freedom in selection of set of the sensors, resulting in a higher degree of control. However, for reliable control, as soon as a point which is not presently clear is scientifically clarified, it is preferable to employ knowledge thereof. Presently, for example, sensors around the hippocampus of the temporal region detect a brain wave relating to the short-term memory, and thus, a fact that highly reliable selection is possible even when graphics representing options are shown not simultaneously but sequentially may be utilized.

When a portion showing an increased blood volume for energy supply to an activity of a group of neurons is detected by irradiating light and detecting reflected/diffused light, it is said that it takes three to five seconds until the blood volume increases after the activity of the neurons has increased. However, the brain wave has an advantage of decreasing the time delay by devising the method of detection.

While the present invention has been described in detail and pictorially in the accompanying drawings, the present invention is not limited to such detail but covers various obvious modifications and equivalent arrangements, which fall within the purview of the appended claims.

What is claimed is:

1. An apparatus for analyzing a brain wave which is installed on a vehicle comprising:
a detection unit for detecting the brain wave signal, and separating and analyzing the detected brain wave signal;
a discrimination unit for generating a control signal according to an intensity of the brain wave signal analyzed by the detection unit;
a processing control unit for controlling subsequent processing according to a type of each of the plurality of control signals produced by the discrimination unit;
at least one brain wave signal induction unit for generating a graphic inducing a predetermined type of the brain wave; and
a display for displaying the generated graphic according to the received signal from the brain wave signal induction unit.

2. The apparatus according to claim 1, wherein the discrimination unit generates control signal by repeating at least two stages in a hierarchy, each corresponding to at most six control signals.

3. The apparatus according to claim 2, wherein the each generation of the plurality of control signal at least two stages is performed by at least two of the brain wave signal induction unit different from each other.

4. The apparatus according to claim 1, further comprising an input unit for controlling a start of an operation of the discrimination unit.

5. The apparatus according to claim 1, wherein the detection unit is installed on one of a ceiling above a seat and a headrest of the seat.

6. The apparatus according to claim 1, wherein the detection unit detects the brain wave without contacting user's head.

7. The apparatus according to claim 1, further comprising a storage unit for storing pattern of the brain wave signal,
wherein the discrimination unit compares the pattern of brain wave signal stored in the storage unit and the brain wave signal detected by the detection unit, and discriminates between a brain wave caused by a primary task such as driving a vehicle and a brain wave caused by controlling an electronic device for a task other than the primary task.

8. The apparatus according to claim 1, further comprising a storage unit for storing pattern of brain wave signal inappropriate for the primary task,
wherein the discrimination unit compares the pattern of brain wave signal stored in the storage unit and the brain wave signal detected by the detection unit, and detects abnormality which includes a state of driving under an influence of alcohol, a state of working under the influence of alcohol, a state of sleeping while driving, a state of sleeping while working, a state of driving while looking aside for a long period, and a state of working while looking aside for a long period.

9. The apparatus according to claim 1, wherein the detection unit comprises an individual difference determination/correction unit for determining and correcting the detected brain wave signal while considering an individual difference thereof to thereby generate a signal used for control.

10. The apparatus according to claim 1, further comprising a brain wave component intensity display unit for generating signal for displaying a component intensity of the brain wave detected by the detection unit on the display.

11. The apparatus according to claim 10, wherein the component intensity of the brain wave is displayed as one of a length of a bar of a bar chart, a size of a graphic, and an area of a graphic.

12. The apparatus according to claim 1, further comprising a BCI training unit,
Wherein the BCI training unit comprises a proficiency determination unit for determining proficiency in operating the apparatus for analyzing a brain wave.

13. The apparatus according to claim 1, further comprising a storage unit,
wherein the storage unit include a first storage unit for storing a brain wave signal of primary task relating to driving, and a second storage unit for storing a brain wave signal of task other than the primary task relating to an operation of the apparatus for analyzing a brain wave.

14. The apparatus according to claim 1, wherein the graphic inducing the predetermined type of the brain wave is a representation partially changed in shape or color.

15. The apparatus according to claim 1, further comprising a shape/color control unit for changing one of a shape and a color of a part close to an edge of display area on the display.

16. The apparatus according to claim 15, wherein the shape/color control unit changes one of a background color, character color, negative/positive inversion, and a representation in a complementary color.

17. The apparatus according to claim 1, further comprising an operation inhibition unit for inhibiting an operation on the apparatus in a case of which processing of the operation requires more steps than a predetermined number of steps.

18. The apparatus according to claim 17, further comprising a BCI training unit,
wherein the BCI training unit comprises a proficiency determination unit for determining proficiency in operating the apparatus for analyzing a brain wave,
wherein the operation inhibition processing unit determines a threshold of the step number to be used for inhibiting the operation based on the proficiency determined by the BCI training unit.

19. The apparatus according to claim 1, detecting an abnormal brain wave signal, and prohibiting driving based on the detected brain wave signal.

20. The apparatus according to claim 19, further comprising a storage unit for storing a pattern of abnormal brain wave signal,
wherein the discrimination unit detects abnormal brain wave signal, by comparing the pattern of abnormal brain wave signal stored in the storage unit and the brain wave signal detected by the detection unit.
